(19) United States
(12) Patent Application Publication
(10) Pub. No.: US 2026/0128146 A1
(43) Pub. Date: May 7, 2026

(54) METHOD, APPARATUS, AND COMPUTER PROGRAM PRODUCT FOR PROVIDING REAL-TIME PRICING INFORMATION AND COMPILATION OF A STANDARDIZED MEASURE

(71) Applicant: McKesson Corporation, Irving, TX (US)

(72) Inventors: Keith Crozier, North Kingstown, RI (US); Stacy McCrommon, Loganville, GA (US); Ashley Proctor, Atlanta, GA (US)

(21) Appl. No.: 19/426,784

(22) Filed: Dec. 19, 2025

Related U.S. Application Data
(63) Continuation-in-part of application No. 17/501,532, filed on Oct. 14, 2021, now Pat. No. 12,525,327.
Application No. 17/501,532 is a continuation-in-part of application No. 16/453,509, filed on Jun. 26, 2019, now Pat. No. 11,562,437.
(63) Continuation-in-part of application No. 17/178,509, filed on Feb. 18, 2021.

(51) Int. Cl. G16H 20/10

(52) U.S. Cl. CPC G16H 20/10

(57) ABSTRACT

A method, apparatus and computer program product are provided for a compiled standardized measure representative of a subset of prescription transactions associated with a controlled substance. The compiled standardized measures may be used in the prescription domain to notify prescribers of a patient's morphine milligram equivalent (MME) to reduce risks related to drug overdose, abuse, and the like. Historical prescription transactions may be accessed to identify prescriptions classified as an opioid or other controlled substance, and that may likely still be in a patient's possession based upon days supply, such that a daily MME may be calculated. Messaging is provided to the prescriber if the amount exceeds a maximum threshold, and the prescription can be changed accordingly.

### CROSS REFERENCES TO RELATED APPLICATIONS

This application is a continuation-in-part of U.S. patent application Ser. No. 17/501,532 filed Oct. 14, 2021, which is a continuation-in-part of U.S. patent application Ser. No. 16/453,509, filed Jun. 26, 2019, the entire contents of which are hereby incorporated by reference in their entireties. This application is a continuation-in part of U.S. patent application Ser. No. 17/178,509, filed Feb. 18, 2021, the entire contents of which are hereby incorporated by reference in its entirety.

### TECHNOLOGICAL FIELD

Embodiments of the present invention relate generally to healthcare transactions and inquiries, and more particularly, to methods, apparatuses, and computer program products for providing real-time pricing information and standardizing an electronic message component.

### BACKGROUND

In the healthcare services industry, patients have difficulty finding pricing information for prescription drugs. In some instances, patients may not be aware of the cost of a prescription until they visit the pharmacy to purchase the prescription. As today's healthcare provider systems continue to evolve, so does the challenge in enabling the patient to understand their out-of-pocket cost. Still further, with developments in cash discount cards and/or systems, in some instances presenting a cash discount card rather than submitting a prescription benefit claim may result in a different cost for the same prescription drug. Additionally, alternative formularies may be available as suitable substitution for a medication, and may be available for a lower cost to the patient in comparison to a prescribed medication, through the benefit plan or a cash discount system. However, a patient typically does not have access to information regarding formulary alternatives and associated costs. Financial structures for prescription claims have become even more sophisticated over time (i.e. formulary tiers, deductibles, maximum benefits, etc.), and prices can vary greatly between pharmacies, making it even more difficult for patients to understand their prescription drugs costs. With the complexities of drug pricing, cash discount systems, insurance pricing agreements, and variation amongst pharmacies, patients particularly have difficulty understanding the costs of the prescriptions.

Additionally, electronic messages pertaining to healthcare transactions are frequently originated, then transmitted and routed according to an identified recipient or destination in the message. A processing system may therefore route the message as directed, monitor a data feed or switch for a response, and forward or route the corresponding response message, once received, to the message originator or requesting computer. In some instances, many messages relating to a similar subject may be transmitted to and from disparate sources that may otherwise not be in direct communication with each other. In this regard, a requesting system may not have access to related messages, nor be able to interpret related messages, initiated by other requesting computers. In some instances, the information within the messages may be useful for some entities to access, but the entity may be unable to access the data due to the separation of information management infrastructure described above, and/or the data may not be in a format useful for the entity.

### BRIEF SUMMARY

Methods, apparatuses, and computer program products are therefore provided for standardizing an electronic message component. Requesting computers, including those requesting prescription pricing information, may generate and transmit messages to a service provider computer that in turn routes the message to an evaluation system, such as a claims processor computer, for evaluation and return of a response to the service provider computer. The service provider computer may then forward the response to the requesting computer. The service provider computer may perform additional edits or manipulation of such messages and responses at various stages of the process, and may further store electronic messages, or historical electronic messages, for subsequent access and/or retrieval.

According to example embodiments provide herein, when the service provider computer receives a prescription inquiry as an electronic message, the service provider computer may access related prescription transactions, such as based on an entity identifier and/or predefined classification of a product identifier (e.g., prescribed medication) identified in the prescription inquiry and the historical transactions. The service provider computer may then process each of the historical transactions to standardize a component of the messages, and compile the standardized data to provide in response to the subject electronic message.

According to certain example embodiments, a prescriber, such as a physician or other medical practitioner may write a prescription for a patient. In some instances, a prescriber may write a prescription for a medication in a special class or a predefined classification, such as an opioid medication. The physician may be unaware of other medication prescribed to the patient, particularly prescriptions written by other healthcare providers, such as a dentist, or specialty provider outside of the prescriber's practice. However, it may be useful for physicians to be aware of other prescriptions for the same patient that fall within the same predefined classification (e.g., opioid medication), to limit or prevent overuse, overdose, addictions, misuse, abuse, and/or the like.

The prescription may be electronically transmitted to a service provider computer for further processing and/or adjudication. According to certain embodiments, the service provider computer may parse the message from the requesting computer (e.g., prescriber computer) to obtain the product identifier (e.g., prescription identifier), and if the prescription has a predefined classification, such as one for which a standardized measure would be useful, the service provider computer accesses historical electronic messages to identify related messages, such as those pertaining to the same predefined classification. The service provider computer can then further process the related historical electronic messages, to standardized measures and compile the measures for transmission to the requesting computer (e.g., prescriber computer). In this regard, the prescriber may receive a summed standardized quantifiable measure per temporal unit for a subject temporal instance. For example, the prescriber may see a morphine milligram equivalents (MME) based on prior prescriptions for the patient, and for one day, or a given day, such as the day the prescriber is entering the prescription (which may be in real-time time or near real-time during a patient visit or patient encounter). The prescriber may therefore be aware of the MME currently prescribed to the patient, even if submitted by other prescribers. An alert can be provided, indicating the compiled standardized measure exceeds a maximum amount per temporal unit (e.g., per day) of a medication associated with a classification (e.g., controlled substance, such as opioids), for a patient. A prescriber may therefore modify the prescription inquiry, and after it is received by the service provider computer, real-time pricing information is provided. Certain time thresholds are used to monitor for a response, and subsequent actions performed according to whether a response is received within the time threshold.

An apparatus is provided, including one or more processors and at least one memory including computer program code that when executed by the one or more processors, causes the one or more processors to perform storing, in a database, (a) a time threshold, (b) a history of prescription transactions comprising respective medications and cash prices, and (c) one or more maximum amounts per temporal unit of a medication associated with a predefined classification that a patient should take. The computer program code, when executed by the one of more processors, further causes the one or more processors to perform receiving from a prescriber computer, a prescription inquiry associated with a patient, a pharmacy, and a medication, and determining the medication is associated with the predefined classification. The computer program code, when executed by the one of more processors, further causes the one or more processors to perform accessing, in the database, prescription transactions associated with the patient and the predefined classification, and the maximum amount per temporal unit of the medication associated with the predefined classification, and determining, based on a days supply of the prescription transactions associated with the patient and the predefined classification, whether respective measures should be included in a compilation.

The computer program code, when executed by the one of more processors, further causes the one or more processors to perform generating a compiled standardized measure representative of a subset of prescription transactions associated with the patient and the predefined classification, by summing the standardized measures per the temporal unit for the prescription transactions.

The computer program code, when executed by the one of more processors, further causes the one or more processors to perform determining the compiled standardized measure exceeds the maximum amount and in response thereto, transmit to the prescriber computer, a response comprising the compiled standardized measure and an alert indicating the prescription would result in the maximum amount being exceeded.

The computer program code, when executed by the one of more processors, further causes the one or more processors to perform receiving a modified prescription inquiry from the prescriber computer, generating a prescription benefit coverage inquiry based on the modified prescription inquiry, and transmitting the prescription benefit coverage inquiry to a pharmacy claims processor computer.

The computer program code, when executed by the one of more processors, further causes the one or more processors to perform monitoring a communication network for receipt of a prescription benefit coverage response associated with the prescription benefit coverage inquiry from the claims processor computer;

[symbol]

The computer program code, when executed by the one of more processors, further causes the one or more processors to perform determining whether a response from the pharmacy claims processor computer is received within the time threshold, and based on a determination that the response from the pharmacy claims processor computer is received from the pharmacy claims processor computer within the time threshold, generating a prescription inquiry response comprising the response from the pharmacy claims processor computer.

The computer program code, when executed by the one of more processors, further causes the one or more processors to perform, based on a determination that the response from the pharmacy claims processor computer is not received within the time threshold, and further based on the stored history of prescription transactions, generating the prescription inquiry response comprising a respective cash price response from the database, and transmitting the prescription inquiry response to the prescriber computer.

According to certain embodiments, the computer program code, when executed by the one of more processors, further causes the one or more processors to perform precluding transmitting the prescription inquiry to the claims processor computer until the prescription inquiry is modified and resubmitted by the prescriber computer.

According to certain embodiments, the computer program code, when executed by the one of more processors, further causes the one or more processors to perform executing a formulary alternative inquiry, wherein executing the formulary alternative inquiry comprises obtaining an alternative formulary response from the database, and transmitting the alternative formulary response to the prescriber computer.

According to certain embodiments, the predefined classification comprises a controlled substance classification, and the temporal unit comprises a number of days. According to certain embodiments, the maximum amount per temporal unit is determined based on at least one of a patient's age, a patient's gender, or a patient's weight. The number of days the compiled standardized measure is applicable is dependent on when one or more prescriptions associated with the prescription transactions is determined to be depleted.

A computer program product comprising at least one non-transitory computer-readable storage medium having computer-executable program code instructions stored therein that when executed by one or more processors, cause the one or more processors to perform storing, in a database, (a) a time threshold, (b) a history of prescription transactions comprising respective medications and cash prices, and (c) one or more maximum amounts per temporal unit of a medication associated with a predefined classification that a patient should take.

The computer-executable program code instructions, when executed by one or more processors, cause the one or more processors to perform receiving from a prescriber computer, a prescription inquiry associated with a patient, a pharmacy, and a medication, and determining the medication is associated with the predefined classification.

The computer-executable program code instructions, when executed by one or more processors, cause the one or more processors to perform accessing, in the database, prescription transactions associated with the patient and the predefined classification, and the maximum amount per temporal unit of the medication associated with the predefined classification, and determining, based on a days supply of the prescription transactions associated with the patient and the predefined classification, whether respective measures should be included in a compilation.

The computer-executable program code instructions, when executed by one or more processors, cause the one or more processors to perform generating a compiled standardized measure representative of a subset of prescription transactions associated with the patient and the predefined classification, by summing the standardized measures per the temporal unit for the prescription transactions, and determining the compiled standardized measure exceeds the maximum amount and in response thereto, transmit to the prescriber computer, a response comprising the compiled standardized measure and an alert indicating the prescription would result in the maximum amount being exceeded.

The computer-executable program code instructions, when executed by one or more processors, cause the one or more processors to perform receiving a modified prescription inquiry from the prescriber computer, generating a prescription benefit coverage inquiry based on the modified prescription inquiry, and transmitting the prescription benefit coverage inquiry to a pharmacy claims processor computer.

The computer-executable program code instructions, when executed by one or more processors, further cause the one or more processors to perform monitoring a communication network for receipt of a prescription benefit coverage response associated with the prescription benefit coverage inquiry from the claims processor computer, and determining whether a response from the pharmacy claims processor computer is received within the time threshold. The computer-executable program code instructions, when executed by one or more processors, further cause the one or more processors to perform based on a determination that the response from the pharmacy claims processor computer is received from the pharmacy claims processor computer within the time threshold, generating a prescription inquiry response comprising the response from the pharmacy claims processor computer.

The computer-executable program code instructions, when executed by one or more processors, cause the one or more processors to perform, based on a determination that the response from the pharmacy claims processor computer is not received within the time threshold, and further based on the stored history of prescription transactions, generating the prescription inquiry response comprising a respective cash price from the database, and transmitting the prescription inquiry response to the prescriber computer.

A computer-implemented method is provided, comprising storing, in a database, (a) a time threshold, (b) a history of prescription transactions comprising respective medications and prices, and (c) one or more maximum amounts per temporal unit of a medication associated with a predefined classification that a patient should take, and receiving from a prescriber computer, a prescription inquiry associated with a patient, a pharmacy, and a medication.

The computer-implemented method includes determining the medication is associated with the predefined classification, accessing, in the database, prescription transactions associated with the patient and the predefined classification, and the maximum amount per temporal unit of the medication associated with the predefined classification, and determining, based on a days supply of the prescription transactions associated with the patient and the predefined classification, whether respective measures should be included in a compilation. The computer-implemented further includes generating a compiled standardized measure representative of a subset of prescription transactions associated with the patient and the predefined classification, by summing the standardized measures per the temporal unit for the prescription transactions, and determining the compiled standardized measure exceeds the maximum amount and in response thereto, transmit to the prescriber computer, a response comprising the compiled standardized measure and an alert indicating the prescription would result in the maximum amount being exceeded.

The computer-implemented method further includes receiving a modified prescription inquiry from the prescriber computer, generating a prescription benefit coverage inquiry based on the modified prescription inquiry, and transmitting the prescription benefit coverage inquiry to a pharmacy claims processor computer. The computer-implemented method further includes monitoring a communication network for receipt of a prescription benefit coverage response associated with the prescription benefit coverage inquiry from the claims processor computer and determining whether a response from the pharmacy claims processor computer is received within the time threshold. The computer-implemented method further includes, based on a determination that the response from the pharmacy claims processor computer is received from the pharmacy claims processor computer within the time threshold, generating a prescription inquiry response comprising the response from the pharmacy claims processor computer.

The method further includes, based on a determination that the response from the pharmacy claims processor computer is not received within the time threshold, and further based on the stored history of prescription transactions, generating the prescription inquiry response comprising a respective cash price from the database, and transmitting the prescription inquiry response to the prescriber computer.

The above summary is provided merely for purposes of summarizing some example embodiments of the invention so as to provide a basic understanding of some aspects of the invention. Accordingly, it will be appreciated that the above described example embodiments are merely examples and should not be construed to narrow the scope or spirit of the disclosure in any way. It will be appreciated that the scope of the disclosure encompasses many potential embodiments, some of which will be further described below, in addition to those here summarized.

### BRIEF DESCRIPTION OF THE SEVERAL VIEWS OF THE DRAWINGS

Having thus described embodiments of the invention in general terms, reference will now be made to the accompanying drawings, which are not necessarily drawn to scale, and wherein:

FIG. 1 is an example overview of a system that can be used to practice some example embodiments described herein;

FIG. 2 is an exemplary schematic diagram of an apparatus in accordance with some example embodiments; and

FIGS. 3 and 4 are flowcharts of operations that may be performed in accordance with some example embodiments.

### DETAILED DESCRIPTION

Some embodiments of the present invention will now be described more fully hereinafter with reference to the accompanying drawings, in which some, but not all embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

As used herein, where a computing device is described herein to receive data from another computing device, it will be appreciated that the data may be received directly from the other computing device and/or may be received indirectly via one or more intermediary computing devices, such as, for example, one or more servers, relays, routers, network access points, and/or the like. Similarly, where a computing device is described herein to transmit data to other computing device, it will be appreciated that the data may be sent directly to the other computing device or may be sent to the other computing device via one or more interlinking computing devices, such as, for example, one or more servers, relays, routers, network access points, and/or the like.

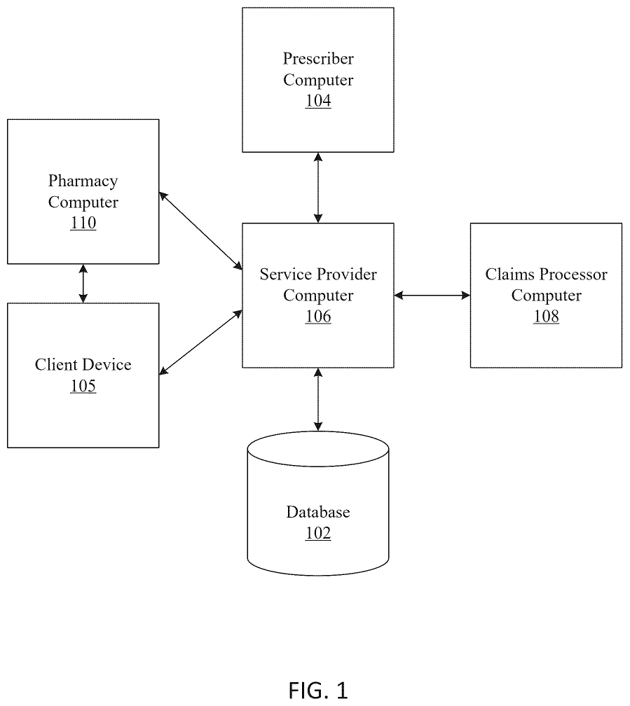

FIG. 1 is an overview of a system that can be utilized to perform operations according to certain example embodiments described herein. The prescriber computer 104 may be optional in certain embodiments, but when present, may be associated with a healthcare provider, such as an entity that may prescribe medication and/or treatments, for example, a physician's office, clinic, long-term care facility, hospital, etc. While the exemplary prescriber computer 104 may be frequently referenced herein as part of a physician's office or healthcare network, the prescriber computer 104 may be associated with any other healthcare provider, such as a hospital, urgent care center, dentist, and/or other medical facility.

The prescriber computer 104, also referred to as a requesting computer, may be any processor-driven device that facilitates the processing of electronic prescription inquiries and/or prescription submission made by physicians or clinical staff, and the communication of information associated with prescription inquiries and/or prescription submission to the service provider computer 106. The execution of the computer-implemented instructions by the prescriber computer 104 may form a special purpose computer or other particular machine that is operable to facilitate the submission of prescription benefit coverage inquiries made by physicians, doctors, clinical staff, pharmacists, and/or the like.

The prescriber computer 104 may therefore facilitate the submission of messages, such as prescription transactions, or prescription requests, and the communication of information associated with such messages to the service provider computer 106. As used herein, the term “prescription request” or “prescription transaction” may refer to any electronic message, such as one transmitted by a prescriber computer that comprises prescription details, such as to inquire regarding compiled standardized measures, as described in further detail herein, and/or to submit a prescription such that a patient can obtain the prescription at a pharmacy.

According to certain embodiments, the requesting computer, such as the prescriber computer 104 may be a device utilized by or in communication with a physician's office, in which a physician or other prescribers enters the details of the prescription, such as during a patient encounter.

The client device 105 may include any user device such as a personal computer, smartphone, laptop, smartwatch, and/or the like. A user of the client device 105, such as a patient, may access the service provider computer 106 (optionally via a service of the pharmacy computer 110 such as a website or mobile “app” of the pharmacy computer 110) to seek pricing information regarding a prescription. The client device 105 may make requests to such a service via a browser, a mobile application, or “app,” operative on the client device 105. In this regard, the client device 105 may be operative as a client device in client-server communications with the pharmacy computer and/or service provider computer 106.

The service provider computer 106 may include, but is not limited to, a processor-driven device that is configured for receiving, processing, and fulfilling prescription inquiries from the client device 105, prescriber computer 104, and/or pharmacy computer 110 regarding prescription pricing and/or other prescription related information. In certain embodiments, a patient may initially access a service of the pharmacy computer 110, which in turn may transmit requests to the service provider computer 106 regarding prescription pricing information.

The service provider computer 106 may be further configured for receiving, processing, and fulfilling inquiries, responses, and/or requests from the client device 105, pharmacy computer 110, prescriber computer 110 and/or the claims processor computer 108, relating to prescription benefit coverage inquiries, prescription tracking, claims processing, benefits, billing, other healthcare transactions, and/or other related activities. Additionally or alternatively, the service provider computer 106 may be operable to facilitate the receipt, routing, and/or processing of prescription inquiries and/or associated responses amongst various components and/or subsystems such as, but not limited to, those depicted in FIG. 1.

In certain exemplary embodiments, the service provider computer 106 may be configured as or may comprise a switch or router that evaluates, processes, modifies, reformats, generates, and/or routes prescription inquiries and/or other healthcare transactions. For example, the service provider computer 106 may route prescription inquiries communicated from the prescriber computer 104, client device 105, and/or pharmacy computer 110 to a claims processor computer 108, such as that associated with a pharmacy benefits manager (PBM), an insurer, a Medicare or other government healthcare insurance program payor, or other payor. According to certain embodiments, the claims processor computer 108 may be referred to as an evaluation system, adjudication computer, and/or payer computer as it evaluates and/or adjudicates prescription inquiries and prescription claims and can facilitate payment of prescription benefit claims.

In certain embodiments, the service provider computer 106 may include a cash price inquiry module configured to generate and/or execute cash price inquiries, such that a cash price response is generated and/or received according to example embodiments and as described in further detail herein. The service provider computer 106 may further include an alternative formulary module configured to generate and/or execute formulary alternative inquiries, such that an alternative formulary response is generated and/or received according to example embodiments and as described in further detail herein. The service provider computer 106 may further compile standardized measures associated with a controlled substance, to determine a prescription would exceed a maximum amount.

In certain embodiments, a message regarding standardized and/or complied measures may be first provided to the prescriber computer, and after the prescriber confirms the prescription, or submits the prescription as final, the service provider computer 106 forwards the transaction for adjudication and provides a response. Various configurations and implementations may be contemplated.

Additionally or alternatively, the service provider computer 106 may reformat prescription inquiries into another form of transaction and modify the recipient information of the reformatted transaction before routing the reformatted transaction to another party, such as a claims processor computer 108. The service provider computer 106 may also direct a prescription inquiry to a claims processor computer 108, which may in turn route a response to the service provider computer 106. The service provider computer 106 may then direct the response to the pharmacy computer 110, client device 105, or other associated entity.

In addition to receiving and storing information, the service provider computer 106 may be further operable to access and/or be in communication with one or more suitable data storage devices, such as a database 102, for storing historical data and/or other various data. In this regard, the database 102 may be referred to as a historical data source. In some embodiments, the database 102 comprises data relating to prescription transactions associated with one or more pharmacy computers 110. Data, such as for example, historical data, may be provided by and/or stored in database 102 by a number of entities which may comprise the prescriber computer 104, service provider computer 106, claims processor computer 108, one or more pharmacy computers 110 and/or other related entities. In certain embodiments, data is provided to database 102 by one or more pharmacy computers 110 associated with one or more pharmacies. These one or more pharmacy computers 110 may voluntarily provide data to database 102 (and/or service provider computer 106, which may in turn store the historical data on database 102), such as historical data related to prior prescription transactions that have taken place at each respective pharmacy. In this regard, the historical data may comprise paid amounts by consumers (e.g., patients) at particular pharmacies for particular prescriptions, and may reflect cash prices (without any insurance payment or coverage), and/or may reflect paid amounts by the consumer given a paid and/or adjudicated prescription claim by the claims processor computer 108. In an embodiment, the one or more pharmacies may be taking part in a program wherein certain data is supplied to database 102 by the one or more pharmacy computers 110 associated with the one or more pharmacies in an effort to provide patients (and prescribers) with accurate cost information. According to some embodiments, the historical data may indicate other characteristics about respective prescription transactions, such as the state or other location information of the dispensing pharmacy, the dispense date, information regarding preauthorization requirements, and/or the like. The service provider computer 106 may be configured to mine and store pertinent information from any healthcare transactions and/or claims received and/or generated by the service provider computer 106, particularly data that may utilized by example embodiments described herein to estimate cost ranges of prescriptions.

The database 102 may include indicators for certain prescription drugs that are controlled substances. For example, the database 102 may track certain classifications of prescription drugs, such as opioid medication. If the product identifier (e.g., prescription identifier, NDC, and/or the like) belongs to a predefined classification. Several different drugs, with different NDCs, can have the same predefined classification. The database 102 may store maximum amounts a patient should consumer within a time period, and the maximum amounts may be based on age, weight, and/or the like.

The database 102 may further include alternative treatment tables that include, without limitation, product identifiers (e.g., national drug code (NDC)), a medication name, a therapeutic class (e.g., RxNorm), or other drug classification, and/or the like. The RxNorm may provide a normalized naming system for generic and branded drugs such that products having the same RxNorm, or therapeutic class, may be considered as formulary alternatives for one another.

In one implementation, each of the alternative treatment tables may be set up at a pharmacy chain/group level based upon data received at least from the pharmacy computers 110. For example, a first alternative treatment table may correspond to a pharmacy chain X, a second alternative treatment table may correspond to a pharmacy chain Y, a third alternative treatment table may correspond to a pharmacy chain Z, etc. Each alternative treatment table may, in one implementation, include claim data received from one or more pharmacy computers associated with each of the pharmacy chains (e.g., pharmacy computer 110). The claim data received from the pharmacy computer may be organized in the one or more alternative treatment tables by RxNorm (e.g., identifying a therapeutic class) and may include, without limitation, one or more product identifiers (e.g., NDC number). In certain embodiments, the alternative treatment tables may include historical data providing an average drug cost (e.g., usual and customary cost) corresponding to each product identifier. Accordingly, accessing the one or more alternative treatment tables may provide information corresponding to a lowest drug cost for multiple products (e.g., NDCs) within a same RxNorm based the upon claim data (e.g., historical claim data) received from the pharmacy computer and stored in database 102. While each of the alternative treatment tables is described as corresponding to a pharmacy chain, it is to be appreciated that the alternative treatment tables may be set up at another group level (e.g., a vendor group level, etc.). Alternative treatment tables are described in further detail in U.S. patent application Ser. No. 15/085,166, filed Mar. 30, 2016, which is hereby incorporated by reference in its entirety.

Accordingly, in addition to and/or instead of providing pricing information related to patient pay amount under a prescription benefit plan and/or cash price, example embodiments may provide alternative formularies and pricing information corresponding thereto, such as under a prescription benefit plan and/or cash discount system.

The service provider computer 106 may transmit responses regarding the prescription inquires to the client device 105 (optionally via the pharmacy computer 110). For example, the service provider computer 106 may notify the pharmacy computer 110 and/or client device 105 of and/or provide a response related to a prescription inquiry from the claims processor computer 108, such as the amount the patient should expect to pay for the prescription at a given pharmacy under a benefit plan, a cash discount system, and/or the like. A response may additionally be provided regarding formulary alternatives.

The example system of FIG. 1 described above is provided merely as an example and it will be appreciated that the example embodiments provided herein may be implemented as or employed by any number of system architectures. Some modifications may be made to certain embodiments. It will be further appreciated that any of the components of FIG. 1 are configured to communicate over a network, or network(s), as described in further detail herein.

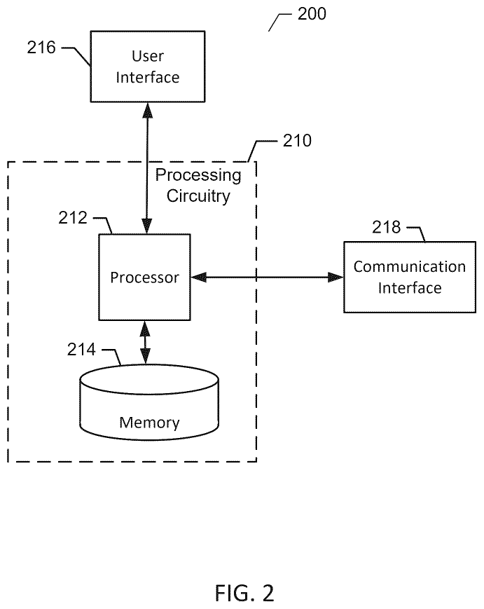

Referring now to FIG. 2, apparatus 200 is a computing device(s) configured for implementing a prescriber computer 104, client device 105, service provider computer 106, pharmacy computer 110, and/or claims processor computer 108, according to example embodiments.

Apparatus 200 may at least partially or wholly embody any of the prescriber computer 104, client device 105, service provider computer 106, pharmacy computer 110, and/or claims processor computer 108. Apparatus 200 may therefore implement any of the prescriber computer 104, client device 105, service provider computer 106, pharmacy computer 110, and/or claims processor computer 108, in accordance with some example embodiments, or may be implemented as a distributed system that includes any of the prescriber computer 104, client device 105, service provider computer 106, pharmacy computer 110, claims processor computer 108, and/or associated network(s).

It should be noted that the components, devices, and elements illustrated in and described with respect to FIG. 2 may not be mandatory and thus some may be omitted in certain embodiments. For example, FIG. 2 illustrates a user interface 216, as described in more detail below, which may be optional in any of the prescriber computer 104, service provider computer 106, pharmacy computer 110, and/or claims processor computer 108. Additionally, some embodiments may include further or different components, devices, or elements beyond those illustrated in and described with respect to FIG. 2.

Continuing with FIG. 2, processing circuitry 210 may be configured to perform actions in accordance with one or more example embodiments disclosed herein. In this regard, the processing circuitry 210 may be configured to perform and/or control performance of one or more functionalities of apparatus 200 in accordance with various example embodiments. The processing circuitry 210 may be configured to perform data processing, application execution, and/or other processing and management services according to one or more example embodiments. In some embodiments apparatus 200, or a portion(s) or component(s) thereof, such as the processing circuitry 210, may be embodied as or comprise a circuit chip. The circuit chip may constitute means for performing one or more operations for providing the functionalities described herein.

In some example embodiments, the processing circuitry 210 may include a processor 212, and in some embodiments, such as that illustrated in FIG. 2, may further include memory 214. The processing circuitry 210 may be in communication with or otherwise control a user interface 216, and/or a communication interface 218. As such, the processing circuitry 210, such as that included in any of the prescriber computer 104, client device 105, service provider computer 106, pharmacy computer 110, claims processor computer 108, and/or apparatus 200 may be embodied as a circuit chip (e.g., an integrated circuit chip) configured (e.g., with hardware, software, or a combination of hardware and software) to perform operations described herein.

The processor 212 may be embodied in a number of different ways. For example, the processor 212 may be embodied as various processing means such as one or more of a microprocessor or other processing element, a coprocessor, a controller, or various other computing or processing devices including integrated circuits such as, for example, an ASIC (application specific integrated circuit), an FPGA (field programmable gate array), or the like. Although illustrated as a single processor, it will be appreciated that the processor 212 may comprise a plurality of processors. The plurality of processors may be in operative communication with each other and may be collectively configured to perform one or more functionalities of apparatus 200 as described herein. The plurality of processors may be embodied on a single computing device or distributed across a plurality of computing devices collectively configured to function as prescriber computer 104, client device 105, service provider computer 106, pharmacy computer 110, claims processor computer 108, and/or apparatus 200. In some example embodiments, the processor 212 may be configured to execute instructions stored in the memory 214 or otherwise accessible to the processor 212. As such, whether configured by hardware or by a combination of hardware and software, the processor 212 may represent an entity (e.g., physically embodied in circuitry - in the form of processing circuitry 210) capable of performing operations according to embodiments of the present invention while configured accordingly. Thus, for example, when the processor 212 is embodied as an ASIC, FPGA, or the like, the processor 212 may be specifically configured hardware for conducting the operations described herein. Alternatively, as another example, when the processor 212 is embodied as an executor of software instructions, the instructions may specifically configure the processor 212 to perform one or more operations described herein.

In some example embodiments, the memory 214 may include one or more non-transitory memory devices such as, for example, volatile and/or non-volatile memory that may be either fixed or removable. In this regard, the memory 214 may comprise a non-transitory computer-readable storage medium. It will be appreciated that while the memory 214 is illustrated as a single memory, the memory 214 may comprise a plurality of memories. The plurality of memories may be embodied on a single computing device or may be distributed across a plurality of computing devices. The memory 214 may be configured to store information, data, applications, computer program code, instructions and/or the like for enabling apparatus 200 to carry out various functions in accordance with one or more example embodiments. For example, when apparatus 200 is implemented as service provider computer 106, memory 214 may be configured to store computer program code for performing corresponding functions thereof, as described herein according to example embodiments.

Still further, memory 214 may be configured to store routing tables, that facilitate determining the destination of communications received from a prescriber computer 104, and/or claims processor computer 108. Memory 214 may further include reconciliation tables for tracking the prescription benefit coverage inquiries received from the prescriber computer 104, and reconciling them with responses received from claims processor computer 108. The memory 214 may be modified as described herein, to store reformatted prescription benefit coverage inquiries with additional information received, determined and/or generated according to example embodiments.

The memory 214 may be further configured to buffer input data for processing by the processor 212. Additionally or alternatively, the memory 214 may be configured to store instructions for execution by the processor 212. In some embodiments, the memory 214 may include one or more databases, such as database 102, that may store a variety of files, contents, or data sets, such as but not limited to historical data and alternative treatment tables. Among the contents of the memory 214, applications may be stored for execution by the processor 212 to carry out the functionality associated with each respective application. In some cases, the memory 214 may be in communication with one or more of the processor 212, user interface 216, and/or communication interface 218, for passing information among components of apparatus 200.

The optional user interface 216 may be in communication with the processing circuitry 210 to receive an indication of a user input at the user interface 216 and/or to provide an audible, visual, mechanical, or other output to the user. As such, the user interface 216 may include, for example, a keyboard, a mouse, a display, a touch screen display, a microphone, a speaker, and/or other input/output mechanisms. As such, in embodiments in which apparatus 200 is implemented as the client device 105, the user interface 216 may, in some example embodiments, provide means for user entry of insurance information, patient information, details relating to a prescription, and/or the like, and for provision of information relating to the cost of a prescription and/or alternative therapies, as described in further detail below. In some embodiments, a user interface 216 may be present in the prescriber computer 104 and/or pharmacy computer 110, such as for a user thereof to enter prescription requests and/or prescription transaction details. In some example embodiments, aspects of user interface 216 may be limited or the user interface 216 may not be present.

The communication interface 218 may include one or more interface mechanisms for enabling communication with other devices and/or networks. In some cases, the communication interface 218 may be any means such as a device or circuitry embodied in either hardware, or a combination of hardware and software that is configured to receive and/or transmit data from/to a network and/or any other device or module in communication with the processing circuitry 210. By way of example, the communication interface 218 may be configured to enable communication amongst any of prescriber computer 104, client device 105, service provider computer 106, pharmacy computer 110, claims processor computer 108, and/or apparatus 200 over a network. Accordingly, the communication interface 218 may, for example, include supporting hardware and/or software for enabling wireless and/or wireline communications via cable, digital subscriber line (DSL), universal serial bus (USB), Ethernet, or other methods.

The network, such as the network in which the system of FIG. 1 or components thereof or components described herein may operate, (e.g., prescriber computer 104, client device 105, service provider computer 106, pharmacy computer 110, claims processor computer 108, and/or apparatus 200, and/or the like) may include a local area network, the Internet, any other form of a network, or in any combination thereof, including proprietary private and semi-private networks and public networks. The network may comprise a wired network and/or a wireless network (e.g., a cellular network, wireless local area network, wireless wide area network, some combination thereof, and/or the like).

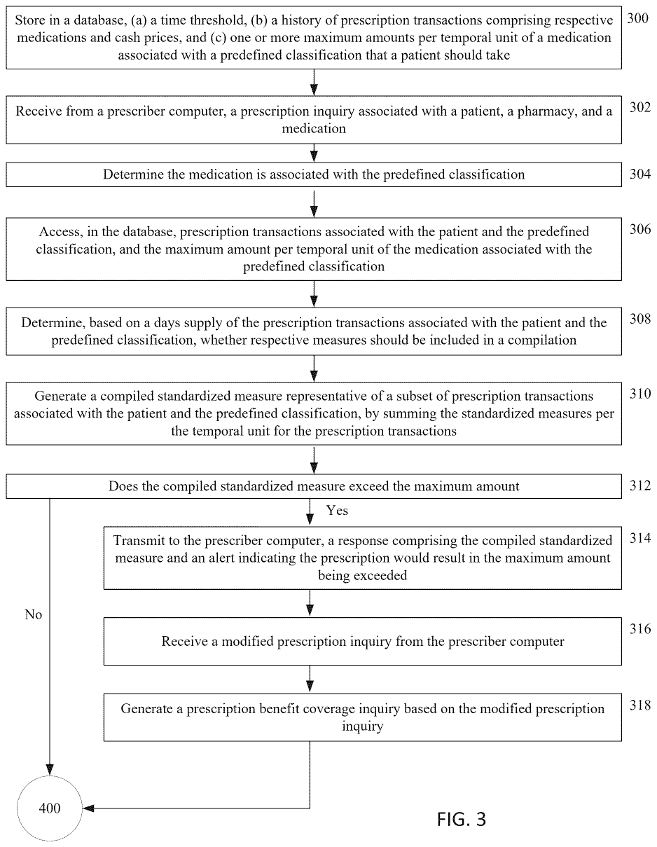

Having now described an example apparatus for implementing example embodiments, FIG. 3 is a flowchart illustrating example operations of an apparatus 200, according to some example embodiments. The operations of FIG. 3 may be performed by apparatus 200, such as with the service provider computer 106 and/or the like.

As shown by operation 300, apparatus 200 may include means, such as processor 212, memory 214, user interface 216, communication interface 218, and/or the like, for storing in a database, such as database 102, (a) a time threshold, (b) a history of prescription claims comprising respective medications and cash prices, and (c) one or more maximum amounts per temporal unit of a medication associated with a predefined classification that a patient should take. As prescription claims are received and processed, the details of the prescription are stored. The prescription claims may comprise a variety of other information, such as, but not limited to: patient demographic information, such as name, date of birth, age, and/or address, insurance/coverage information such as cardholder name, cardholder ID, member ID and/or other identifier, bank identification number (BIN), group ID and/or group Information, prescriber information such as Primary Care Provider ID or other identifier (e.g. NPI code), Primary Care Provider Name, Prescriber ID or other identifier (e.g. NPI code, DEA number), patient's Preferred Pharmacy or other Healthcare Provider Information (e.g. store name, chain identifier, store address, etc.), various claim information such as drug, service, or product information (e.g. via NDC number), Prescription/Service Reference Number, Date Prescription Written, Diagnosis/Condition, Number of Refills Authorized, and/or the like. In this regard a history of prescription claims is built and expanded on in the database as additional prescription claims are received and processed.

The database 102 further stores the time threshold, including a time threshold for which the service provider computer 106 will await a response from a claims processor computer before attempting an alternative pricing method, for example. The time thresholds may be initially configured via a user interface and stored in the database.

The database 102 further stores the one or more maximum amounts per temporal threshold unit of a medication associated with a predefined classification that a patient should take. A maximum amount per temporal threshold (e.g., per day) unit may be an MME, and/or other threshold. The maximum amounts can be configured by a user interface and stored in database 102. The maximum amounts may serve as a threshold or guideline for which to alert prescribers of corresponding risks for the patient, when the maximum amount is met or exceeded, as described in further detail herein.

As shown by operation 302, apparatus 200 may include means, such as processor 212, memory 214, user interface 216, communication interface 218, and/or the like, for receiving from a prescriber computer, a prescription inquiry associated with a patient, a pharmacy, and a medication. The prescription inquiry may be received as a message from a prescriber computer such as in response to a prescriber entering prescription details during a patient encounter and/or the like, and can include any prescription details listed herein such as those described with respect to operation 300.

For example, the prescription inquiry may include a product identifier, such as a prescription identifier identifying a drug. The message, prescription request, and/or prescription transaction may further comprise a temporal parameter such a number of days supply, or “days supply” of the prescription drug, such as a 30-day supply. The message may further include an identifier of an insurance provider and/or corresponding claims processor computer (e.g., evaluation system, PBM and/or adjudication computer).

The prescription inquiry, including the information described above, may be transmitted via a communication interface 218 (and optionally via a pharmacy computer 110) to the service provider computer 106.

According to certain embodiments, the service provider computer 106 may receive a plurality of prescription inquiries, such as from one or more client devices 105 and/or pharmacy computers 110, on a continual and/or ongoing basis and may process such requests as described in further detail herein, in real-time or near real-time. The term “near” real-time is used to express that the prescription inquiry may be processed, and the pricing information, described in further detail below, may be desired within a fraction of a second, or seconds, from the time the prescription inquiry is submitted, to account for computer processing delays.

As shown by operation 304, apparatus 200 may include means, such as processor 212, memory 214, user interface 216, communication interface 218, and/or the like, for determining the medication is associated with the predefined classification. For example, the processor 212 may determine the medication is associated with a classification as opioid. The determination of whether the product identifier of the subject electronic message is associated with a predefined classification may be performed by the service provider computer 106, in real-time and/or near real-time relative to receiving the message from the prescriber computer 104.

As shown by operation 306, apparatus 200 may include means, such as processor 212, memory 214, communication interface 218, and/or the like, for accessing, in the database, prescription transactions associated with the patient and the predefined classification, and the maximum amount per temporal unit of the medication associated with the predefined classification. The predefined classification may be any groupings of a product such as a prescription drug, defined in memory 214 such as database 102. For example, the service provider computer 106 may track or access certain classifications of prescription drugs, such as opioid medication. If the product identifier (e.g., prescription identifier, NDC, and/or the like) associated with a medication indicated in the prescription inquiry belongs to a predefined classification, the service provider computer 106 may access other prescriptions for the patient reflecting prescription drugs having the same predefined classification as the subject electronic message.

As shown by operation 308, apparatus 200 may include means, such as processor 212, memory 214, communication interface 218, and/or the like, for determining, based on a days supply of the prescription transactions associated with the patient and the predefined classification, whether respective measures should be included in a compilation. The historical electronic messages (e.g., prescriptions) that should be included in the compilation, such as active prescriptions, may be identified in a variety of ways, including using a temporal measure associated with a respective message. The temporal measure may indicate a days supply of a prescription, such as 30 days. In this regard, example embodiments may make certain assumptions regarding prescription adherence, and determine that if the temporal measure (e.g., days supply) has lapsed, based on a comparison of the current date, and a date a prescription was obtained from a pharmacy by a patient (or written by a prescriber), the patient is no longer taking the prescription and the respective historical electronic message can be discarded for the purpose of compiling the standardized measures.

If, on the other hand, it can be determined the patient is likely still taking the prescription, or could be taking the prescription, or may have the prescription in their possession, such as by determining the number of days that have passed since the day the prescription was obtained or written is within the number of days supply, the respective historical electronic message should be included in the compilation. Certain variations may be contemplated. For example, as a precaution, certain embodiments may buffer or pad the temporal measure, such as by a predetermined number of days, and/or percentage number of days of the days supply, incase a patient draws out a prescription longer than the days supply.

As shown by operation 310, apparatus 200 may include means, such as processor 212, memory 214, communication interface 218, and/or the like, for generating a compiled standardized measure representative of a subset of prescription transactions associated with the patient and the predefined classification, by summing the standardized measures per the temporal unit for the prescription transactions.

The standardized measure per temporal unit may be based on dosage and quantity per day, and may be standardized as a total weight (e.g., in milligrams) of the drug taken per day (or other temporal unit). Accordingly, processor 212 of example embodiments may calculate a total weight per day as the standardized measure for a respective historical electronic message (e.g., prescription). Many variations may be contemplated. For example, if a prescription request or prescription transaction indicates a range of doses per day, such as taking a dose every 4-6 hours, example embodiments may be configured to use a minimum amount, average amount, or maximum amount the patient would take when following the prescription. As yet another example, a minimum amount and maximum amount may be separately tracked such that standardized measures described in further detail below are provided as ranges.

Because example embodiments utilize a classification to identify related prescriptions, the prescription used in the compilation may therefore need not necessarily be associated with the same prescription drug, but rather the same predetermined classification, such as an opioid or other classification, such as medication classified as a controlled substance. This provides an extra level of precaution as some drugs that don't necessarily have the same NDC may be risky to take together, or risky at or above certain maximum amounts for the respective controlled substance.

The standardized measure per temporal units are added together to determine a compiled standardized measure. In the scenario of a classification of opioid medication, the compiled standardized measure may reflect a morphine milligram equivalent (MME), such as an MME per day that a patient may be taking.

Many modifications may be contemplated. For example, a sum may be calculated as a running sum each time a historical prescription is identified as relating to the subject electronic message (e.g., associated with the same patient). Additionally or alternatively, a standardized measure per temporal unit for the prescription inquiry may be further incorporated into the compiled standardized measure such that the compiled standardized measure also reflects the prescription inquiry if the associated prescription was filled.

As shown by operation 312, apparatus 200 may include means, such as processor 212, memory 214, communication interface 218, and/or the like, for determining whether the compiled standardized measure exceeded the maximum amount. If so, at operation 314, apparatus 200 may include means, such as processor 212, memory 214, communication interface 218, and/or the like, for transmitting to the prescriber computer, a response comprising the compiled standardized measure and an alert indicating the prescription would result in the maximum amount being exceeded.

According to certain embodiments, the processor 212 may preclude transmitting the prescription inquiry to the claims processor computer 108 until the prescription inquiry is modified and resubmitted by the prescriber computer. Accordingly, message sent to the claims processor computer 108, for prescriptions only to be later cancelled and reversed, are reduced and conserve computer and network resources associated with the service provider computer 106 and the claims processor computer 108.

According to certain embodiments, an alternative formulary inquiry may be generated by an alternative formulary inquiry module of the service provider computer 106 and provided to the prescriber computer. The alternative formulary inquiry may be generated in accordance with an application programming interface (API) of an alternative formulary inquiry module, for example. In some embodiments, the alternative formulary inquiry may be a specialized data object configured for obtaining formulary alternatives (and/or corresponding pricing information), and may comprise any data needed from the prescription inquiry to determine formulary alternatives (and/or corresponding pricing information), such as but not limited to the medication (e.g., NDC), pharmacy at which the medication is to be obtained, and/or the like. The formulary alternative inquiry (e.g., the formulary alternative data object) may therefore be generated and stored on memory 214 for further processing as described below with respect to operation 312.

In certain embodiments, executing the alternative formulary inquiry may be performed by the alternative formulary inquiry module of the service provider computer 106. Example embodiments, such as processor 212, may query the one or more alternative treatment tables, such as those stored on database 102, to determine whether an alternative treatment and/or alternative product equivalent for the medication is available. In certain embodiments, the query may be based upon the pharmacy, or service provider identifier, indicated in the prescription inquiry. In one implementation, example embodiments may search for an alternative treatment table corresponding to the pharmacy name field in the prescription inquiry, populated with a short pharmacy name. The apparatus 200 may query the identified alternative treatment table for the RxNorm and/or NDC identified in the prescription inquiry and/or associated therewith. In certain examples, the query may include further search parameters or filtering, based on patient payment threshold, usual and customary costs, and/or the like, as described in further detail in Ser. No. 15/085,166.

The response to the prescriber computer 104 may include, in addition to the compiled standardized measure, additional information such as the prescriptions or other information from the historical electronic messages included in the compilation, descriptions of risks associated with the predetermined classification and/or compiled standardized measure, and/or the like, such as risks associated with opioid medications. In certain examples, a recommended range, or recommended maximum amount per temporal unit (e.g., per day) may be provided. The recommendation may be tailored, such as by taking into account the patient's age, gender, weight, and/or the like. The edited message and/or response message may further comprise additional information, such as the number of days the compiled standardized measure may be applicable for the patient. For example, if one or more prescriptions used in the compilation is determined to be depleted in a day or a few day, this information may be useful to the prescriber.

The edited message and/or response message may further comprise alternative medications and/or the like. As yet another example, a recommended day or time in the future on which the patient should begin taking the prescription may be provided, and a prescriber may write a prescription accordingly. For example, the response may recommend the prescription is not filled until a specified date, for example, 5 days in the future, or provide a “do not fill before” date. Additionally, information such as pricing, estimated pricing, and/or the like may also be provided.

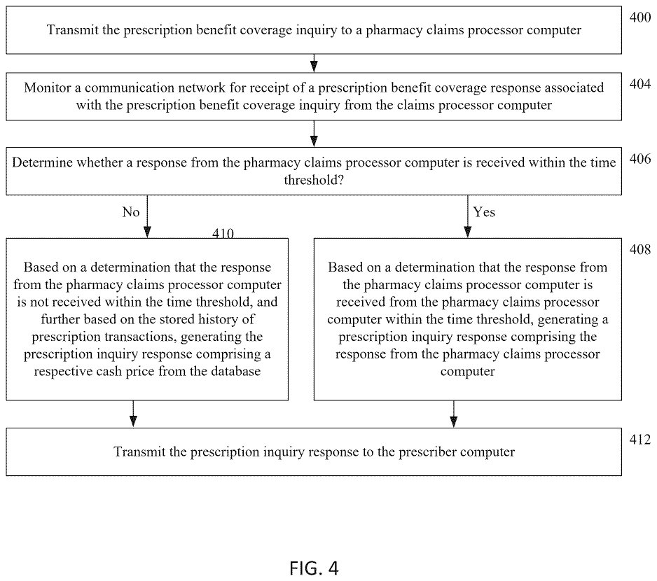

If the maximum amount is not exceeded at 312, processing may continue to operation 400 of FIG. 4.

Following operation 314, the prescriber computer receives the response including the compiled standardized measure and the alert regarding the prescription and the maximum amount exceeded with regard to the controlled substance. According to certain embodiments, an alternative formulary can be provided. A prescriber can review the response via a user interface, and may modify the prescription via the user interface. A prescriber may therefore be notified that a prescription may result in a patient having too high of an amount of a controlled substance, and may be notified of associated risks such as addiction and abuse.

The prescriber may be compelled to change the prescribed medication, dosage, days supply, and/or the like. The apparatus 200 can therefore provide a user interface enabling such modification and resubmission of the prescription inquiry. In operation 316 apparatus 200 may include means, such as processor 212, memory 214, communication interface 218, and/or the like, for receiving the modified prescription inquiry from the prescriber computer.

In operation 318, apparatus 200 may include means, such as processor 212, memory 214, communication interface 218, and/or the like, for generating a prescription benefit coverage inquiry based on the modified prescription inquiry and processing may continue to operation 400 of FIG. 4.

Turning to FIG. 4, in operation 400, apparatus 200 may include means, such as processor 212, memory 214, communication interface 218, and/or the like, for transmitting the prescription benefit coverage inquiry to a pharmacy claims processor computer. The transmitted prescription benefit coverage inquiry may be based on the modified prescription inquiry from operation 318 or an unmodified prescription inquiry, if not modified by the prescriber computer as set forth according to operations 314, 316 and 318.

According to certain embodiments, apparatus 200 determines a claims processor computer 108 to which to transmit the prescription benefit inquiry such as by accessing routing tables stored on memory 214 (such as database 102) to determine a network address and/or other information enabling data communication with a claims processor computer 108 associated with the user's, or patient's, prescription benefit plan. Any of the insurance information included in prescription inquiry, such as but not limited to a BIN, member ID, cardholder ID and/or other identifier, group ID and/or group information may be utilized to determine which claims processor computer, potentially from a plurality of claims processor computers to which transmit and/or route the prescription benefit inquiry.

The prescription benefit inquiry may be a specialized data object configured for obtaining patient pay amounts under a prescription benefit plan. In some examples, the prescription benefit inquiry may be in accordance with one or more predefined and/or standardized formats, such as the National Council for Prescription Drug Programs (NCPDP), and/or the like. The prescription benefit inquiry may include any information from the prescription inquiry, such as those required by NCPDP and/or other standardized format, such as but not limited to any information in the prescription inquiry. The prescription benefit inquiry may include more data fields in comparison to a cash price inquiry. For example, the prescription benefit inquiry may include, in addition to the NDC or other medication identifying information, numerous other data related to the prescription benefit plan, such as a member ID, group ID, BIN, and/or other data fields.

In operation 404, apparatus 200 may include means, such as processor 212, memory 214, communication interface 218, and/or the like, for monitoring a network for receipt of a prescription benefit coverage response associated with the prescription benefit coverage inquiry from the claims processor computer. As the service provider computer 106 may function a switch in data communication with a plurality of claims processor computers 108 (and plurality of prescriber computers 104, pharmacy computers 110, and client devices 105), the service provider computer 106 may receive numerous responses, on an ongoing basis, over the network and communication interface 218.

Accordingly, when such responses are received, example embodiments, such as processor 212, access one or more reconciliation tables and/or other data stored that enables the processor 212 to reconcile responses with their corresponding requests (e.g., prescription inquiries). In certain scenarios, if a timeout with no response associated with the inquiry is received within the time threshold (e.g., 2 seconds), a prescription inquiry response may be generated and/or stored indicating no response from the claims processor computer was received. Alternative operations may be performed in such examples, such as calculating price estimates according to historical prices paid under the benefit plan.

In operation 408, apparatus 200 may include means, such as processor 212, memory 214, communication interface 218, and/or the like, for, based on a determination that the response from the pharmacy claims processor computer is received from the pharmacy claims processor computer within the time threshold, generating a prescription inquiry response comprising the response from the pharmacy claims processor computer. The response may therefore include the price a patient will pay for the prescription.

If it is determined at 406 that a response is not received within the time threshold, at operation 410, apparatus 200 may include means, such as processor 212, memory 214, communication interface 218, and/or the like, for based on a determination that the response from the pharmacy claims processor computer is not received within the time threshold, and further based on the stored history of prescription transactions, generating the prescription inquiry response comprising a respective cash price from the database. In this regard, if a response is not received regarding the patient's prescription benefit plan and price to be paid for the prescription under the prescription benefit plan, the patient and/or prescriber can be notified of a cash price (e.g., insurance coverage) for which the prescription can be obtained.

According to certain embodiments, the cash price inquiry may be generated by a cash price inquiry module of the service provider computer 106. The cash price inquiry may be generated in accordance with an application programming interface (API) of a cash price inquiry module, for example. According to certain embodiments, the cash price inquiry may be a specialized data object configured for obtaining cash prices and/or cash price estimates of certain medication, and may comprise any of the data needed from the prescription inquiry to obtain the cash price and/or cash price estimate, such as but not limited to the medication (e.g., NDC), pharmacy at which the medication is to be obtained, and/or the like. The cash price inquiry (e.g., the cash price inquiry data object) may therefore be generated and stored on memory 214 for further processing.

In certain embodiments, executing the cash price inquiry may be performed by the cash price inquiry module of the service provider computer 106 and/or invoking an associated API. Executing the cash price inquiry may include executing certain computer program code configured to access historical cash prices and/or cash transaction history associated with the pharmacy to estimate a cash price for the prescribed medication at the pharmacy indicated in the prescription inquiry. Example embodiments may access the historical data, such as from the database 102. According to certain example embodiments, the service provider computer 106 may store and utilize historical data, such as historical cash prices from prior prescription transactions that were transmitted from the pharmacy computer 110 to the service provider computer 106. For example, the pharmacy computer 110 may work in agreement with the service provider computer 106 to provide the data regarding cash purchases made at the pharmacy. In this regard, example embodiments may query the database 102 to determine a cash discount system that provides the lowest patient pay amount for the prescription, in comparison to other cash discount systems. A BIN or other identifying information of a cash discount system may be included in the response. The particular cash discount system indicated in the cash price response may be identified from a plurality of cash discount systems.

In certain embodiments, historical information may be stored in database 102 in association with a particular pharmacy and/or pharmacy chain, and/or state. For example, various claims processor computers 108 may provide pricing of certain drugs for sale by a particular pharmacy and/or pharmacy chain within a certain state. Accordingly, the historical data evaluated may be specifically associated with the pharmacy and/or pharmacy chain indicated in the prescription inquiry. Additionally or alternatively, historical information evaluated may be limited to a particular time frame, such as the prior 2 weeks or prior one month. This may ensure that more recent data is evaluated to increase the chances of the historical data still being applicable to current transactions. Further detail regarding cash price inquiries and the potential cash price responses is provided in U.S. patent application Ser. No. 16/867,286, filed May 5, 2020, which is hereby incorporated by reference in its entirety.

The service provider computer 106 may maintain the historical information and evaluate the historical data, to determine the best cash price for the patient, and generate the cash price response such that the cash price response includes the price and associated cash discount system. In certain examples, additional information may be evaluated in the cash price response, such as pharmacy economic information. The pharmacy economic information, or revenue, may be utilized to determine if the cash price response should be provided to the patient.

In certain embodiments, the claims processor computer 108 to which an inquiry is submitted may be operated separately from the service provider computer 106 and may adjudicate or otherwise process prescription benefit coverage inquiries received from various sources, such as but not limited to the service provider computer 106. In some embodiments, the adjudication may comprise a determination of whether the medication associated with the prescription benefit coverage inquiry is covered by the patient's insurance and may provide additional medication cost information relevant to the patient and/or prescriber.

In operation 412, apparatus 200 may include means, such as processor 212, memory 214, communication interface 218, and/or the like, for transmitting the prescription inquiry response to the prescriber computer. Whether the prescription inquiry response includes a patient pay amount under a prescription benefit plan (returned by the claims processor computer) or a cash price, the prescriber and patient can have a discussion around affordability which can improve adherence and ensure the patient follows through with obtaining the prescription medication.

According to certain embodiments, the apparatus 200 generating a cash price inquiry, a formulary alternative inquiry, and a prescription benefit inquiry based on the prescription inquiry. Each of the cash price inquiry, a formulary alternative inquiry, and a prescription benefit inquiry may be in a different format than that of the prescription inquiry, and may be in different formats from each other.

Numerous variations of the illustrated and disclosed operations and processes may be contemplated. According to certain embodiments, operations relating to obtaining a cash price, a price paid under a prescription benefit plan, and/or a formulary alternative may occur simultaneously, or in any order, without dependency on each other. Accordingly, different sub-processes, modules, and/or circuitry of the service provider computer 106 may perform such operations. According to certain embodiments, a cash price may be obtained and provided regardless of whether a response from the claims processor computer is received within the time threshold. According to certain embodiments, both a cash price and patient pay amount under a benefit plan may be provided to the prescriber computer 104. According to certain embodiments, alternative formularies may also be provided to the prescriber computer 104.

In this regard, in certain examples, a single response may be generated that includes any and/or all of the relevant pricing information to be returned to the patient via the client device 105. As used herein, the prescription inquiry response may be additionally or alternatively referred to as a consolidated prescription inquiry response. For example, the prescription inquiry response may compile and/or consolidate data relating to the patient pay amount returned by the claims processor computer 108, a cash price obtained in the cash price response, and/or a formulary alternative and associated pricing. The prescription inquiry response may therefore be in a different format than any of the prescription benefit coverage inquiry, the cash price response and/or the alternative formulary response, because the prescription inquiry response is configured to incorporate data from any or all of the prescription benefit coverage inquiry, the cash price response and/or the alternative formulary response. The prescription inquiry response may, however, be in a predefined format defined by the service provider computer 106, such that the prescription inquiry response may be processed and displayed by a device. In this regard, a prescription benefit coverage response, a cash price response, and an alternative formulary response may be reformatted into a prescription inquiry response (e.g., single prescription inquiry response).

In certain scenarios of missing and/or erroneous data, such as one in which a prescription benefit coverage response is not received from the claims processor, alternative operations may be performed, such as obtaining price estimates based on historical data. Additionally or alternatively, a message may be inserted indicating certain price checks are inconclusive or unavailable.

Example embodiments provide technical improvements to such systems by implementing the apparatus 200 such that the pricing information is provided to the patient in real-time or near real-time as the physician prescribes a medication for a patient. The term “near” real-time is used to express that the response is provided at the prescriber computer 104 with only short computer or network processing times, such as within a fraction of a second, or seconds, from the time the prescription benefit coverage inquiry is submitted,. The technical challenges in providing this real-time feedback are increased by the evolving complexities of healthcare transactions and their associated processing by claims processing computers 108, and the uncertainty or inconsistency of response quality and response time associated therewith. The challenges are further increased by the ever increasing volume of data received from pharmacies and/or claims processors. Example embodiments leverage the historical data in a manner described herein that provides efficient, and accurate pricing for patients and physicians at the point of prescription.

The solutions provided by example embodiments therefore improve the usage of processing resources, and additionally or alternatively improve the functioning of the service provider computer 106 by reducing and/or eliminating erroneous responses and reducing and/or eliminating instances in which patients are left without cost information when responses are not provided by a claims processor computer 108.

Similarly, example embodiments may reduce and/or eliminate the need for prescription inquiry resubmissions, and/or the like, caused by users such as prescribers not understanding why a response was not received, and resubmitting identical prescription benefit coverage inquiries in hopes of receiving a proper response. Having various sources of prescription pricing information may increase the probability that a valid price and/or price estimate is returned, therefore reducing or limiting the need for resubmission of inquiries. This may therefore reduce the resources expended, such as memory and/or processing power, that may otherwise be required to facilitate the resubmission (and possibly numerous resubmissions) of the same or similar prescription inquiries, as well as the associated rerouting, and reprocessing of the resubmitted transaction(s) throughout the various components described herein. Likewise, example embodiments may reduce processing resources otherwise expended on extensive research, custom queries, and/or the like, when cost estimates for prescriptions cannot otherwise be obtained. Accordingly, example embodiments described herein further improve the technical efficiency of systems implementing and/or employing such embodiments.

Example embodiments provided herein further provide improvements to the technology of electronic healthcare transaction and data management. A variety of electronic healthcare data relating to patients is stored in disparate systems, but many practitioners may not have access to such data due to the division of different medical practices, specialties, and/or the like. Example embodiments leverage prescription claim data in an unconventional and non-routine way, including utilizing data relating to other prescribers, to provide compiled standardized measures, such as MME, associated with a controlled substance, in real-time or near real-time. Example embodiments therefore enable prescribers to affect change in the medication they prescribe patients to reduce or prevent drug abuse, misuse, overdose, and/or the like, which may save lives and/or reduce addiction.

Example embodiments therefore provide distinct advantages over alternative solutions, such as applications providing similar information at the pharmacy when the patient obtains the prescription. In such examples, a pharmacist may raise concern relating to prescriptions but may have to contact a prescriber to affect change to the drug therapy, as a pharmacist is not typically able to change a prescription, and/or attempt to counsel a patient in the pharmacy when the patient attempts to obtain the prescription, which may be ineffective. Mail order prescriptions may result in missed opportunities for counseling abuse altogether. However, according to example embodiments, when a prescriber is alerted of a potential issue by viewing the compiled standardized measure in real-time or near real-time upon entering the prescription, the prescriber can choose alternative drugs and/or therapy to prescribe, change dosage, and/or counsel the patient.

Example embodiments may further provide technical improvements by conserving resources otherwise expended to route, process, then cancel electronic messages for prescriptions sent to a pharmacy and in some cases for prescription claim adjudication, but for which the medication is not acquired due to pharmacist recommendations and/or counseling at the pharmacy. In this regard, earlier intervention and potential prescription modification can be made by the prescriber after the prescriber is notified, and before additional messages are sent to a claims processor computer requesting pricing information. Providing the compiled standardized measure to the prescriber in real-time or near real-time may therefore reduce the number of prescriptions (and associated electronic transactions) that are sent to a claims processor computer and/or pharmacy computer but later cancelled and/or reversed, such as when a pharmacist intervenes, which may occur to risks associated with controlled substances. Accordingly, restocking at the pharmacy, and the associated computational transactions and expense associated with prescription claim reversal may be reduced, such that computer resource consumption and network consumption is reduced, and the efficiency of the overall system, the service provider computer 106, claims processor computer 108, and the pharmacy computer 110 is improved.

It will be appreciated that the figures are each provided as examples and should not be construed to narrow the scope or spirit of the disclosure in any way. In this regard, the scope of the disclosure encompasses many potential embodiments in addition to those illustrated and described herein. Numerous other configurations may also be used to implement embodiments of the present invention.

FIGS. 3 and 4 illustrate operations of a method, apparatus, and computer program product according to some example embodiments. It will be understood that each operation of the flowchart or diagrams, and combinations of operations in the flowchart or diagrams, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which embody the procedures described herein may comprise one or more memory devices of a computing device (for example, memory 214) storing instructions executable by a processor in the computing device (for example, by processor 212). In some example embodiments, the computer program instructions of the computer program product(s) which embody the procedures described above may be stored by memory devices of a plurality of computing devices. As will be appreciated, any such computer program product may be loaded onto a computer or other programmable apparatus (for example, apparatus 200) to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product may comprise an article of manufacture which implements the function specified in the flowchart block(s). The computer program instructions of one or more computer program products may also be loaded onto a computer or other programmable apparatus (for example, apparatus 200 and/or other apparatus) to cause a series of operations to be performed on the computer or other programmable apparatus to produce a computer-implemented process such that the instructions which execute on the computer or other programmable apparatus implement the functions specified in the flowchart block(s).

Accordingly, blocks of the flowchart support combinations of means for performing the specified functions and combinations of operations for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. An apparatus comprising one or more processors and at least one memory including computer program code that when executed by the one or more processors, causes the one or more processors to perform:
storing, in a database, (a) a time threshold, (b) a history of prescription transactions comprising respective medications and cash prices, and (c) one or more maximum amounts per temporal unit of a medication associated with a predefined classification that a patient should take;
receiving from a prescriber computer, a prescription inquiry associated with a patient, a pharmacy, and a medication;
determining the medication is associated with the predefined classification;
accessing, in the database, prescription transactions associated with the patient and the predefined classification, and the maximum amount per temporal unit of the medication associated with the predefined classification;
determining, based on a days supply of the prescription transactions associated with the patient and the predefined classification, whether respective measures should be included in a compilation;
generating a compiled standardized measure representative of a subset of prescription transactions associated with the patient and the predefined classification, by summing the standardized measures per the temporal unit for the prescription transactions;
determining the compiled standardized measure exceeds the maximum amount and in response thereto, transmit to the prescriber computer, a response comprising the compiled standardized measure and an alert indicating the prescription would result in the maximum amount being exceeded;
receiving a modified prescription inquiry from the prescriber computer;
generating a prescription benefit coverage inquiry based on the modified prescription inquiry;
transmitting the prescription benefit coverage inquiry to a pharmacy claims processor computer;
monitoring a communication network for receipt of a prescription benefit coverage response associated with the prescription benefit coverage inquiry from the claims processor computer;
determining whether a response from the pharmacy claims processor computer is received within the time threshold;
based on a determination that the response from the pharmacy claims processor computer is received from the pharmacy claims processor computer within the time threshold, generating a prescription inquiry response comprising the response from the pharmacy claims processor computer;
based on a determination that the response from the pharmacy claims processor computer is not received within the time threshold, and further based on the stored history of prescription transactions, generating the prescription inquiry response comprising a respective cash price response from the database; and
transmitting the prescription inquiry response to the prescriber computer.

2. The apparatus according to claim 1, wherein the computer program code, when executed by the one of more processors, further causes the one or more processors to perform:
precluding transmitting the prescription inquiry to the claims processor computer until the prescription inquiry is modified and resubmitted by the prescriber computer.

3. The apparatus according to claim 1, wherein the computer program code, when executed by the one of more processors, further causes the one or more processors to perform:
executing a formulary alternative inquiry, wherein executing the formulary alternative inquiry comprises obtaining an alternative formulary response from the database; and
transmitting the alternative formulary response to the prescriber computer.

4. The apparatus according to claim 1, wherein the predefined classification comprises a controlled substance classification.

5. The apparatus according to claim 1, wherein the temporal unit comprises a number of days.

6. The apparatus according to claim 1, wherein the maximum amount per temporal unit is determined based on at least one of a patient's age, a patient's gender, or a patient's weight.

7. The apparatus according to claim 1, wherein a number of days the compiled standardized measure is applicable is dependent on when one or more prescriptions associated with the prescription transactions is determined to be depleted.

8. A computer program product comprising at least one non-transitory computer-readable storage medium having computer-executable program code instructions stored therein that when executed by one or more processors, cause the one or more processors to perform:
storing, in a database, (a) a time threshold, (b) a history of prescription transactions comprising respective medications and cash prices, and (c) one or more maximum amounts per temporal unit of a medication associated with a predefined classification that a patient should take;
receiving from a prescriber computer, a prescription inquiry associated with a patient, a pharmacy, and a medication;
determining the medication is associated with the predefined classification;
accessing, in the database, prescription transactions associated with the patient and the predefined classification, and the maximum amount per temporal unit of the medication associated with the predefined classification;
determining, based on a days supply of the prescription transactions associated with the patient and the predefined classification, whether respective measures should be included in a compilation;
generating a compiled standardized measure representative of a subset of prescription transactions associated with the patient and the predefined classification, by summing the standardized measures per the temporal unit for the prescription transactions;
determining the compiled standardized measure exceeds the maximum amount and in response thereto, transmit to the prescriber computer, a response comprising the compiled standardized measure and an alert indicating the prescription would result in the maximum amount being exceeded;
receiving a modified prescription inquiry from the prescriber computer;
generating a prescription benefit coverage inquiry based on the modified prescription inquiry;
transmitting the prescription benefit coverage inquiry to a pharmacy claims processor computer;
monitoring a communication network for receipt of a prescription benefit coverage response associated with the prescription benefit coverage inquiry from the claims processor computer;
determining whether a response from the pharmacy claims processor computer is received within the time threshold;
based on a determination that the response from the pharmacy claims processor computer is received from the pharmacy claims processor computer within the time threshold, generating a prescription inquiry response comprising the response from the pharmacy claims processor computer;
based on a determination that the response from the pharmacy claims processor computer is not received within the time threshold, and further based on the stored history of prescription transactions, generating the prescription inquiry response comprising a respective cash price from the database; and
transmitting the prescription inquiry response to the prescriber computer.

9. The computer program product according to claim 8, wherein the computer-executable program code instructions, when executed by one or more processors, cause the one or more processors to perform:
precluding transmitting the prescription inquiry to the claims processor computer until the prescription inquiry is modified and resubmitted by the prescriber computer.

10. The computer program product according to claim 8, wherein the computer-executable program code instructions, when executed by one or more processors, cause the one or more processors to perform:
executing a formulary alternative inquiry, wherein executing the formulary alternative inquiry comprises obtaining an alternative formulary response from the database; and
transmitting the alternative formulary response to the prescriber computer.

11. The computer program product according to claim 8, wherein the predefined classification comprises a controlled substance classification.

12. The computer program product according to claim 8, wherein the temporal unit comprises a number of days.

13. The computer program product according to claim 8, wherein the maximum amount per temporal unit is determined based on at least one of a patient's age, a patient's gender, or a patient's weight.

14. The computer program product according to claim 8, wherein a number of days the compiled standardized measure is applicable is dependent on when one or more prescriptions associated with the prescription transactions is determined to be depleted.

15. A computer-implemented method comprising:
storing, in a database, (a) a time threshold, (b) a history of prescription transactions comprising respective medications and prices, and (c) one or more maximum amounts per temporal unit of a medication associated with a predefined classification that a patient should take;
receiving from a prescriber computer, a prescription inquiry associated with a patient, a pharmacy, and a medication;
determining the medication is associated with the predefined classification;
accessing, in the database, prescription transactions associated with the patient and the predefined classification, and the maximum amount per temporal unit of the medication associated with the predefined classification;
determining, based on a days supply of the prescription transactions associated with the patient and the predefined classification, whether respective measures should be included in a compilation;
generating a compiled standardized measure representative of a subset of prescription transactions associated with the patient and the predefined classification, by summing the standardized measures per the temporal unit for the prescription transactions;
determining the compiled standardized measure exceeds the maximum amount and in response thereto, transmit to the prescriber computer, a response comprising the compiled standardized measure and an alert indicating the prescription would result in the maximum amount being exceeded;
receiving a modified prescription inquiry from the prescriber computer;
generating a prescription benefit coverage inquiry based on the modified prescription inquiry;
transmitting the prescription benefit coverage inquiry to a pharmacy claims processor computer;
monitoring a communication network for receipt of a prescription benefit coverage response associated with the prescription benefit coverage inquiry from the claims processor computer;
determining whether a response from the pharmacy claims processor computer is received within the time threshold;
based on a determination that the response from the pharmacy claims processor computer is received from the pharmacy claims processor computer within the time threshold, generating a prescription inquiry response comprising the response from the pharmacy claims processor computer;
based on a determination that the response from the pharmacy claims processor computer is not received within the time threshold, and further based on the stored history of prescription transactions, generating the prescription inquiry response comprising a respective cash price from the database; and
transmitting the prescription inquiry response to the prescriber computer.

16. The computer-implemented method according to claim 15, further comprising:
precluding transmitting the prescription inquiry to the claims processor computer until the prescription inquiry is modified and resubmitted by the prescriber computer.

17. The computer-implemented method according to claim 15, further comprising:
executing a formulary alternative inquiry, wherein executing the formulary alternative inquiry comprises obtaining an alternative formulary response from the database; and
transmitting the alternative formulary response to the prescriber computer.

18. The computer-implemented method according to claim 15, wherein the predefined classification comprises a controlled substance classification.

19. The computer-implemented method according to claim 15, wherein the temporal unit comprises a number of days.

20. The computer-implemented method according to claim 15, wherein the maximum amount per temporal unit is determined based on at least one of a patient's age, a patient's gender, or a patient's weight.
